(19) United States
(12) Patent Application Publication
(10) Pub. No.: US 2004/0092895 A1
(43) Pub. Date: May 13, 2004

(54) Soft contact tip for use with a hand-held debridement device

(76) Inventor: Kim R. Harmon, Mineral City, OH (US)

Correspondence Address: BAKER & DANIELS, 111 E. WAYNE STREET, SUITE 800, FORT WAYNE, IN, 46802

(21) Appl. No.: 10/289,610

(22) Filed: Nov. 7, 2002

(51) Int. Cl. A61M 35/00

(52) U.S. Cl. 604/289

(57) ABSTRACT

A soft contact debridement tip for use with hand-held wound debridement devices. The soft contact debridement tip of the present invention provides concurrent irrigation and suction to the area to be debrided. The soft contact debridement tip includes a foam end having an opening for providing irrigation and suction to the area to be debrided. Advantageously, the foam end of the present invention facilitates the removal of excess fluid at the debridement site, and at the same time alleviates pain and discomfort associated with wound debridement. The soft contact tip of the present invention is adapted for single use, and is easily attached and detached from a hand-held debridement device.

### BACKGROUND OF THE INVENTION

1. Field of the Invention

This invention relates generally to hand-held wound debridement devices, and, more particularly, to a soft contact tip for such devices.

2. Description of the Related Art

Debridement is the surgical excision of dead, devitalized, and contaminated tissue, and/or the removal of foreign matter from a wound. Debridement is advantageous because it cleanses the wound and lowers the bacterial count of wound tissues, thereby facilitating the healing process. Hand-held wound debridement devices generally include an irrigation system to provide pressurized streams of fluid to dislodge unwanted tissue and foreign matter from a wound and a suction system to collect the dislodged matter and irrigation fluid. Hand-held wound debridement systems which utilize pressurized streams of fluid to dislodge unwanted tissue and foreign matter from a wound generally provide an efficient and effective means of debriding a wound. However, the use of such systems can, disadvantageously, result in the splashing of irrigation fluid and body fluids from the patient onto, e.g., the healthcare professional operating the debridement device.

Developments have been made to reduce the amount of splash generated from wound debridement devices utilizing irrigation fluid as the mechanism for debridement. Various shields or splash guards have been utilized in an effort to deflect excess irrigation and body fluids away from the healthcare professional operating the debridement device. Conventional splash deflection devices generally comprise structures formed about and extending outwardly from a debridement tip. These prior art devices suffer from numerous deficiencies. For example, the suction created by the debridement tip typically forms a sufficient vacuum to cause conventional splash deflection shields to adhere to the patient during the debridement procedure, resulting in ineffective irrigation of the wound and patient discomfort.

One conventional method of solving the problem of conventional splash shields vacuum adhering to patients provides one or more vent holes in the portion of the splash shield extending outwardly from the debridement tip. The vent holes of prior art devices are radially displaced from the debridement tip and seek to prevent the shield from adhering to the patient by preventing the formation of a vacuum between the shield and the patient. Problematically the vent holes of conventional splash shields allow irrigation fluids and body fluids to seep and even spray out of the vent holes, thereby causing a marked decrease in the effectiveness of these prior art splash shields.

Wound debridement devices generally include a debridement tip for providing concurrent fluid and suction to the area to be debrided. Conventionally, the debridement tip is manufactured out of a hard plastic material which, when inserted into or placed in contact with the wound being debrided, can cause patient discomfort.

What is needed in the art is a debridement tip which effectively provides concurrent irrigation and suction to the area to be debrided, and which, minimizes patient discomfort.

Also needed in the art is a debridement splash shield which is effective in reducing the amount of splash generated from wound debridement devices utilizing irrigation fluid as the mechanism of debridement and which has a decreased tendency to suction adhere to the patient undergoing debridement.

### SUMMARY OF THE INVENTION

The present invention is a soft contact debridement tip for use with hand-held debridement devices, such as the various Zimmer Pulsavac® hand-held debridement devices. The soft contact tip of the present invention includes a proximal adapter for engaging a mating portion of a hand-held debridement device and a distal tip for insertion into or contact with the wound being debrided. Advantageously, the distal tip of the soft contact tip of the present invention is formed from a resilient and absorbent material such as an open celled foam material. Owing to the material of construction, the soft contact tip of the present invention can be maneuvered over and inside a wound being debrided with reduced discomfort to the patient. Advantageously, the debridement tip of the present invention can be a single use, disposable item, and may be easily attached to and detached from the debridement device.

In one embodiment of the present invention, the soft contact tip of the present invention comprises a foam element having a toroidal shape supported on a foam support pad. Irrigation and suction is delivered to the area to be debrided through the central opening of the toroidal foam element.

The soft contact debridement tip of the present invention is advantageous for numerous reasons. For instance, the flexible nature of the foam end makes it capable of conforming to irregularly shaped wounds. This capability results in more efficient debridement, and at the same time, reduces the spray of irrigation and/or body fluids from the area being debrided. Also, the absorbent nature of the foam end allows it to absorb excess fluid at the debridement site, thereby resulting in a more efficient debridement procedure. Furthermore, the air permeable nature of the foam end makes the soft contact tip of the present invention less susceptible to vacuum adhering to a patient during the debridement procedure. However, one or more vent holes can be added to a contact tip of the present invention for even greater resistance to vacuum adhering to a patient during the debridement procedure. A further advantage of the present invention is that the foam end requires less contact force on the wound being debrided to achieve adequate fluid removal from the wound site. Furthermore, the foam end of the soft contact tip of the present invention can effect mechanical debridement as the foam end is maneuvered over the wound being debrided. Stated another way, the foam end will dislodge unwanted tissue and foreign matter from a wound as it is moved over the wound, i.e., when the wound is scrubbed with the foam end.

An object of the present invention is to provide a soft contact debridement tip which effectively provides concurrent irrigation and suction to the area to be debrided, and which minimizes patient discomfort.

A further object of the present invention is to provide a debridement tip having an integral splash shield effective in reducing the amount of splash generated from debridement devices utilizing irrigation fluid as mechanism for debridement and which does not have a tendency to suction adhere to the patient undergoing debridement.

### BRIEF DESCRIPTION OF THE DRAWINGS

The aforementioned and other features and objects of this invention, and the manner of attaining them, will become more apparent and the invention itself will be better understood by reference to the following description of an embodiment of the invention taken in conjunction with the accompanying drawings, wherein:

FIG. 1 is a perspective view of a hand-held wound debridement device equipped with the soft contact debridement tip of the present invention; and

FIG. 2 is a cross-sectional view of the soft contact debridement tip shown in FIG. 1.

Corresponding reference characters indicate corresponding parts throughout the several views. Although the drawings represent an embodiment of the present invention, the drawings are not necessarily to scale and certain features may be exaggerated in order to better illustrate and explain the present invention. The exemplification set out herein illustrates an embodiment of the invention, in one form, and such exemplification is not to be construed as limiting the scope of the invention in any manner.

### DETAILED DESCRIPTION OF THE PRESENT INVENTION

The embodiments disclosed below are not intended to be exhaustive or to limit the invention to the precise form disclosed in the detailed description. Rather, the embodiments are chosen and described so that others skilled in the art might utilize their teachings.

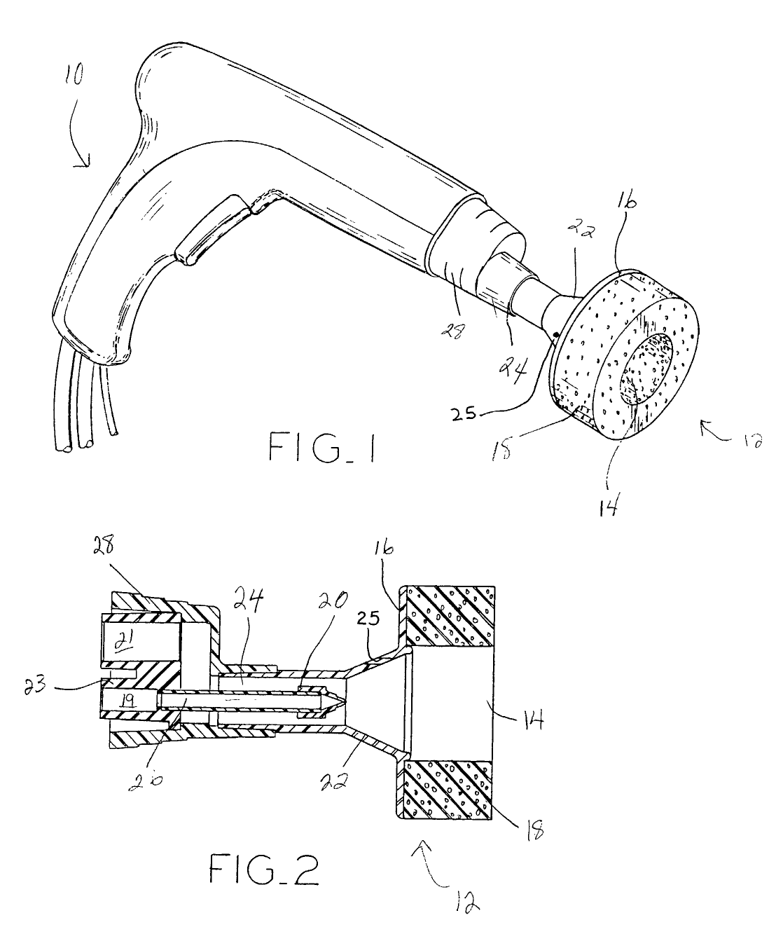

With reference to FIGS. 1 and 2, hand-held wound debridement device 10 is equipped with soft contact debridement tip 12 of the present invention. Soft contact tip 12 includes fitting 28 adapted to couple soft contact tip 12 to hand-held wound debridement device 10. Soft contact debridement tip 12 is designed for single use, and adapter housing 28 may be easily attached and detached from adapter housing 30, as necessary.

Fitting 28 includes fluid port 19 and suction port 21 adapted to couple soft contact tip 12 to a source of irrigation fluid and suction, respectively. Fluid port 19 and suction port 21 are part of unitary structure 23, which is connected to fitting 28 via, e.g., sonic welds. Suction port 21 is in fluid communication with suction passage 24 (discussed hereinafter), and is independent from fluid port 19.

Fluid port 19 includes fluid passage 26 fitted with spray tip 20. Fluid passage 26, in conjunction with spray tip 20, is adapted to provide the desired supply of irrigation fluid to the area to be debrided. Spray tip 20, as illustrated in FIG. 2, can be adapted to emit irrigation fluid in a variety of spray patterns including, e.g., a fan spray.

Fluid passage 26 extends from fitting 28 and through suction passage 24. Suction passage 24 and fluid passage 26 are independent of one another, and provide concurrent irrigation and suction to the area being debrided. Suction passage 24 extends radially to form cone adapter 22. Foam support pad 16 is attached to cone adapter 22, and supports foam end 18. In one exemplary embodiment, cone adapter 22 includes one or more vent holes 25. Foam end 18 includes opening 14 for providing irrigation and suction to the area being debrided. The porous quality of foam end 18 makes it capable of absorbing excess fluid from the wound. Additionally, the use of foam end 18 reduces the pain and discomfort associated with inserting debridement tip 18 into or onto the wound.

In one exemplary embodiment, foam end 18 is formed of an open celled foam. One acceptable open celled foam material is the willsorb-WH material available from illbruck, Inc. The willsorb-WH material is a white, fine pore, ester polyurethane foam capable of absorbing and holding fluid as well as transferring fluid. This material is soft-pliable when dry and dimensionally stable. The willsorb-WH material has a density of about 1.8 lbs./cubic foot, and a pore size of about 85 ppi (pores per linear inch). The willsorb-WH material has been tested according to ASTM D-3574 and determined to have a dry tensile strength of about 30 psi, and a wet tensile strength of about 25 psi. Furthermore, the willsorb-WH material has been found, according to ASTM D-3574, to have a dry tear of about 4.2 lbs/linear inch, about 400% elongation when dry, about 480% elongation when wet, and a volume swell of about 3%.

While this invention has been described as having an exemplary design, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed:

1. A debridement tip having a proximal end and a distal end, the proximal end being adapted for connection to an irrigation source and a suction source, the debridement tip comprising:
a fitting adjacent the proximal end, the fitting including an irrigation port and a suction port for connecting the debridement tip to the irrigation source and the suction source, respectively;
an irrigation passage connected in fluid communication with said irrigation port, said irrigation passage operable to convey a quantity of irrigation fluid from said irrigation port to the distal end of the debridement tip;
a suction passage connected in fluid communication with said suction port, said suction passage operable to convey a quantity of suction from said suction port to the distal end of the debridement tip; and
a foam pad extending from the distal end of the debridement tip.

2. The debridement tip of claim 1, wherein said foam pad comprises a toroidal foam pad having an open center, and wherein said suction and said irrigation traverse said open center of said toroidal foam pad.

3. The debridement tip of claim 1, further comprising:
a foam support pad connected to the distal end of the debridement tip and supporting said foam pad.

4. The debridement tip of claim 1, wherein said suction passage includes a vent hole.

5. The debridement tip of claim 1, wherein said foam pad is formed from an open celled foam material.

6. A debridement tip having a proximal end and a distal end, the debridement tip comprising:
delivery means for delivering a quantity of irrigation fluid and a quantity of suction to an area to be debrided; and
resilient contact means for resiliently contacting the area to be debrided.

7. The debridement tip of claim 6, wherein said resilient contact means comprises a foam pad extending from the distal end of the debridement tip.

8. The debridement tip of claim 7, wherein said foam pad comprises a toroidal foam pad having an open center.

9. The debridement tip of claim 7, wherein said foam pad is formed from an open celled foam material.

10. The debridement tip of claim 7, further comprising:
a foam support pad connected to the distal end of the debridement tip and supporting said foam pad.

11. A method of debriding a wound, the method comprising the steps of:
providing a hand-held debridement device;
providing a soft contact debridement tip adapted for attachment to the hand-held debridement device, the soft contact debridement tip including a foam end;
attaching the soft contact debridement tip to the debridement device;
placing the foam end of the soft contact debridement tip adjacent the wound; and
debriding the wound.

12. The method of claim 11, wherein said step of debriding the wound comprises:
applying irrigation fluid and suction to the wound.

13. The method of claim 12, wherein said step of debriding the wound further comprises:
scrubbing the wound with the foam end of the soft contact debridement tip.
